(19) United States
(12) Patent Application Publication
(10) Pub. No.: US 2022/0257445 A1
(43) Pub. Date: Aug. 18, 2022

(54) Normobaric Hypoxia Trainer

(71) Applicant: United States of America as represented by the Secretary of the Navy, Patuxent River, MD (US)

(72) Inventors: Rocco Portoghese, Winter Park, FL (US); Matthew Adams, Orlando, FL (US); Tyson Griffin, Oviedo, FL (US); Nelson Lerma, Orlando, FL (US); Chad Farwig, Oviedo, FL (US)

(21) Appl. No.: 17/176,467

(22) Filed: Feb. 16, 2021

(51) Int. Cl. A61G 10/02; G09B 9/08; A63B 23/18

(52) U.S. Cl. CPC A61G 10/02; A63B 23/18; G09B 9/085

(57) ABSTRACT

A normobaric hypoxia trainer including a training chamber, an intake fan for allowing ground level air to be introduced into the training chamber; an exhaust fan for removing air from the training chamber; a plurality of circulation fans for mixing interior air of the training chamber to create a uniform oxygen concentration within the training chamber; a nitrogen generation system, the nitrogen generating system including a plurality of polysulphone membrane cartridges for separating out nitrogen from air; a compressor for supplying compressed air to the nitrogen generation system; a pressure regulator for regulating the pressure of the compressed air; a heater for controlling temperature of the compressed air, the heated pressure regulated compressed air passing through the polysulphone membrane cartridges such that nitrogen can be separated out from the air; and, a flow controller for controlling flow rate of the separated nitrogen into the training chamber.

### STATEMENT OF GOVERNMENT INTEREST

The invention described herein may be manufactured and used by or for the Government of the United States of America for governmental purposes without payment of any royalties thereon or therefor.

### BACKGROUND

Hypoxia training has long been performed in hypobaric chambers or enclosures, which are rooms from which air is removed to create the low-pressure conditions encountered at altitude. Hypobaric chambers are absolutely realistic but come with an array of mechanical challenges and physiological dangers. The concept of normobaric hypoxia training was developed to avoid the problems associated with low pressure chambers. In normobaric hypoxia training oxygen is removed or displaced to create low-oxygen conditions inside the chamber with physiological effects similar to low air pressures without actually changing the air pressure in the training environment.

There are many methods of removing or displacing oxygen from an environment. The simplest method is to displace room air by introducing nitrogen or low-oxygen air from storage tanks. This method, however, requires the presence and handling of high-pressure tanks. A leak anywhere in the system can create unexpected and dangerous hypoxic conditions outside the training chamber. In addition, long-term costs are increased. Industry recognizes that creating nitrogen on demand has lower long-term operating costs than sourcing nitrogen from compressed gas suppliers. Finally, releasing nitrogen from storage tanks can only increase effective altitude, while the method includes no mechanism for decreasing effective altitude. A separate system or method is required to return oxygen to the environment in order to bring the chamber to a lower simulated altitude.

Creating nitrogen of a controlled concentration on-demand can be accomplished in several ways through different technologies. Some currently available devices produce hypoxic conditions inside a chamber by drawing air from the training chamber, removing oxygen, and returning the now lower-oxygen air to the chamber. Some outside air must be admixed into the training chamber to replace the volume of the discarded oxygen to maintain normobaric conditions. This creates inefficiencies by reintroducing a portion of the oxygen just removed. In addition, recycling the majority of the air in the chamber places an absolute limit on the total amount of time that can be spent in training as the carbon dioxide exhaled by the trainees inevitably begins to build up to dangerous levels. This effect worsens as the number of enclosed trainees increase. Eventually training must be discontinued and all air in the chamber purged.

An alternative method is to remove oxygen from ambient air by some method and introduce this low-oxygen air into the training chamber, constantly purging the training chamber. Normobaric conditions are preserved simply by allowing air to escape the chamber through passive venting. The constant introduction of new air into the chamber prevents carbon dioxide build up. The method of purging an environment to maintain a set concentration of a gas, called “sweep-purge,” is well known. However, currently available industrial oxygen concentration control systems are not well-suited to aviation training. They do not provide an ability to change oxygen concentration set points, to change simulated altitudes, or to change set points in a controlled manner to emulate flight profiles. Additionally, existing system do not intelligently or quickly react to external forcing functions, such as the introduction of oxygen to the training environment, an inevitable occurrence in human training as trainees go on and off recovery air as part of their hypoxia recovery training.

### SUMMARY

The present invention is directed to a method for providing a normobaric hypoxia trainer that meets the needs enumerated above and below.

The present invention is directed to a normobaric hypoxia trainer wherein the normobaric hypoxia trainer includes a training chamber, an intake fan for allowing ground level air to be introduced into the training chamber, an exhaust fan for removing air from the training chamber, a plurality of circulation fans for mixing interior air of the training chamber to create a uniform oxygen concentration within the training chamber, a nitrogen generation system, a compressor for supplying compressed air to the nitrogen generation system, a pressure regulator for regulating the pressure of the compressed air, a heater for controlling temperature of the compressed air, and a flow controller. The nitrogen generating system includes a plurality of membrane cartridges. The compressed air passes through the membrane cartridges such that nitrogen can be separated out from the air, and the flow controller controls the flow rate of the separated nitrogen into the training chamber.

It is a feature of the invention to provide a normobaric hypoxia trainer that is well suited to aviation and military training.

It is a feature of the invention to provide a normobaric hypoxia trainer that can change oxygen concentration set points, change simulated altitudes, and/or change set points in a controlled manner to emulate flight profiles.

It is a feature of the present invention to provide a normobaric hypoxia trainer that intelligently and quickly reacts to external forcing functions, such as the introduction of oxygen to the training environment.

### DRAWINGS

These and other features, aspects and advantages of the present invention will become better understood with reference to the following description and appended claims, and accompanying drawings wherein:

FIG. 1 is a view of the normobaric hypoxia training system;

FIG. 1A is a view of the internal components of the nitrogen generation system;

FIG. 2 is a flowchart describing the altitude control algorithm high level state determination decision tree;

FIG. 3 is a flowchart describing the altitude control algorithm emergency descent mode operation;

FIG. 4 is a flowchart describing the altitude control algorithm increase oxygen concentration/decrease simulated altitude operation;

FIG. 5 is a flowchart describing the altitude control algorithm decrease oxygen concentration/increase simulated altitude operation, and;

FIGS. 6A and 6B is a flowchart describing the altitude control algorithm maintain oxygen concentration/maintain altitude operation.

### DESCRIPTION

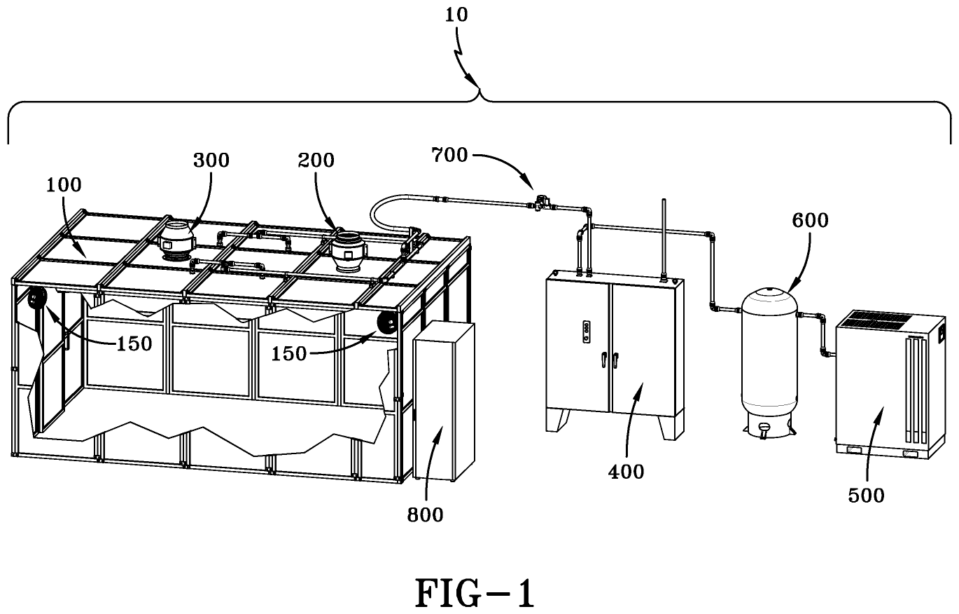

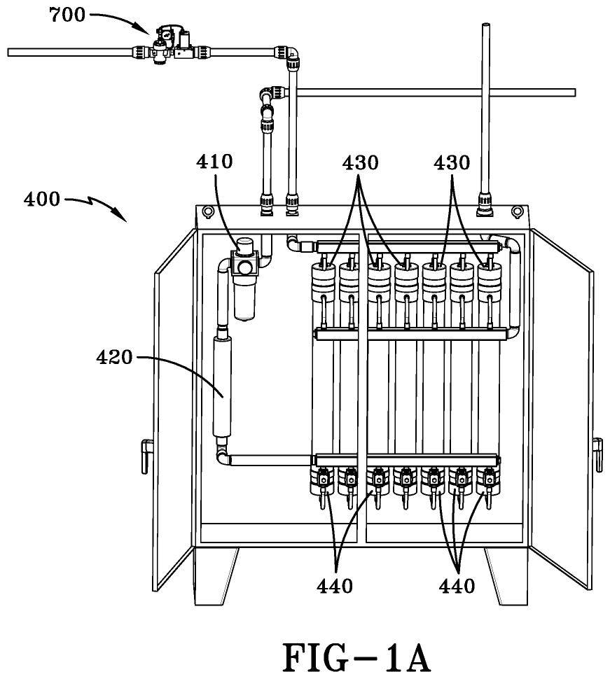

The preferred embodiments of the present invention are illustrated by way of example below and in FIGS. 1-6. As shown in FIG. 1, the normobaric hypoxia trainer 10 includes a training chamber 100, an intake fan 200 for allowing ground level air to be introduced into the training chamber 100, an exhaust fan 300 for removing air from the training chamber 100, a plurality of circulation fans 150 for mixing interior air of the training chamber 100 to create a uniform oxygen concentration within the training chamber 100, a nitrogen generation system 400, a compressor 500 for supplying compressed air to the nitrogen generation system 400, a receiver tank 600 to store compressed air, and a flow controller 700. As shown in FIG. 1A, the nitrogen generating system 400 includes a pressure regulator 410 for regulating the pressure of the compressed air, a heater 420 for controlling temperature of the compressed air, a plurality of polysulphone membrane cartridges 430, each controlled by its own pneumatic solenoid valve 440. The compressed air passes through the polysulphone membrane cartridges 430 such that nitrogen can be separated out from the air, and the flow controller 700 controls the flow rate of the separated nitrogen into the training chamber 100. The operation of the system is monitored and controlled by a controller 800.

In the description of the present invention, the invention will be discussed in a military environment; however, this invention can be utilized for any type of application related to hypoxia training.

In one of the preferred embodiments, as shown in FIG. 1A, there are seven polysulfone membrane cartridges 430 plumbed in parallel. To be plumbed in parallel can be defined, but without limitation, as the cartridges 430 being pneumatically connected such that all share the same air inflow and outflow points.

The polysulfone membranes cartridges 430 act as molecular filters. In operation, when a pressure differential is created across a membrane cartridge 430, oxygen, water vapor, and other gases readily pass through the membrane material while nitrogen does not, separating the gases. The nitrogen is collected while the other gases are discarded. Although polysulphone is the discussed membrane, the membrane can be manufactured from any material that performs the functions outlined.

In the general case, the nitrogen purity of air output by the polysulfone membrane cartridges 430 can be controlled by changing the air pressure entering the membrane within the cartridge 430, air and membrane temperature, flow rate through the membrane cartridges 430, and the number of cartridges 430 in use. The percentage of oxygen passed through a polysulfone membrane cartridge 430 increases as the pressure across the membrane cartridge 430 increases. The oxygen permittivity of the membrane cartridge 430 increases as air and membrane cartridge 430 temperature increases. The membrane cartridge's 430 effectiveness increases as the flow rate through the cartridge 430 decreases and air spends a longer amount of time within the cartridge 430. Since the cartridges 430 in the array are plumbed in parallel, increasing the number of cartridges 430 in use while flow through the array of polysulfone membrane cartridges 430 as a whole is held constant, reduces the flow rate through each cartridge 430 and increases their effectiveness.

The pressure of the air fed to the plurality of cartridges 430 is held constant at the cartridges' 430 recommended operating pressure by the pressure regulator 410. The heater 420 is used to heat the air to the membrane cartridges 430 recommended operating temperature. A control system (or controller) 800 controls nitrogen purity by selecting the number of cartridges 430 in use and controlling the airflow rate through the cartridges. Individual cartridges can be added to or removed from the parallel array by remotely controlled pneumatic solenoid valves 440. The flow rate through the cartridge array is controlled by a flow controller 700 placed after the cartridges 430.

In the preferred embodiment, temperature, pressure, flow rate, oxygen and carbon dioxide sensors distributed throughout the nitrogen generation system 400 and training chamber 100 allow the control system 800 to monitor the process and training environment at all times, providing inputs to a controlling algorithm. In the preferred embodiment the algorithm is Altitude Control Algorithm. In addition, the operator is provided with an emergency stop button that can be used to cease training and rapidly return the training chamber 100 to the ground normal oxygen concentration.

The Altitude Control Algorithm (ACA) is responsible for reaching and/or maintaining the operator's intended simulated altitude. The ACA may run on a programmable logic controller. In operation, the algorithm constantly monitors system sensors and operator inputs to determine the current state of the environment inside the training chamber 100 and move it toward the operator's intended simulated altitude. Inputs to the ACA include the current oxygen concentration of the training chamber 100, the target oxygen concentration of the training environment as determined by the operator's current altitude setting, the current oxygen concentration in the nitrogen generation system 400 outflow and the current state of the emergency stop button. Outputs from the ACA are the membrane cartridges 430 to be used, the desired air flow rate, the speed of the chamber intake fan 200 and the speed of the chamber exhaust fan 300.

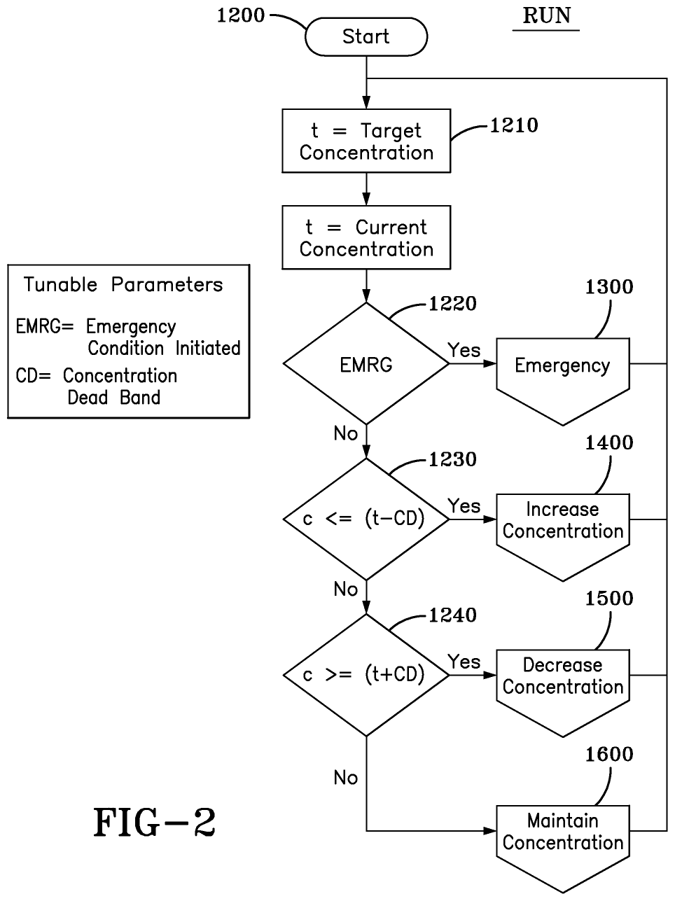

During operation, the ACA is always in one of four states as determined by operator input and the current and target training environment oxygen concentrations. The high-level ACA state determination flowchart is shown in FIG. 2. The algorithm starts at block 1200. Target oxygen concentration 1210 and current training environment oxygen concentration 1215 values are sampled. If the emergency stop button has been pressed 1220 the system enters the emergency descent state 1300. Otherwise, an oxygen concentration dead band value 1230 is used to determine if the target oxygen concentration has been reached. The dead band value 1230 is defined as part of system setup; the ACA uses a value that represents a few hundred feet of elevation change at the median altitude to be simulated. If the current training environment oxygen concentration is less than or equal to the target concentration minus the dead band value 1230, the algorithm enters increase concentration (descending altitude) state 1400. If the concentration is greater than or equal to the target concentration plus the dead band value 1240, the algorithm enters the decrease concentration (ascending altitude) state 1500. If none of these conditions are true, then the training chamber 100 is within the dead band value 1230 of the target concentration and is considered to be at the desired concentration, therefore the algorithm enters maintain concentration (level flight) state 1600.

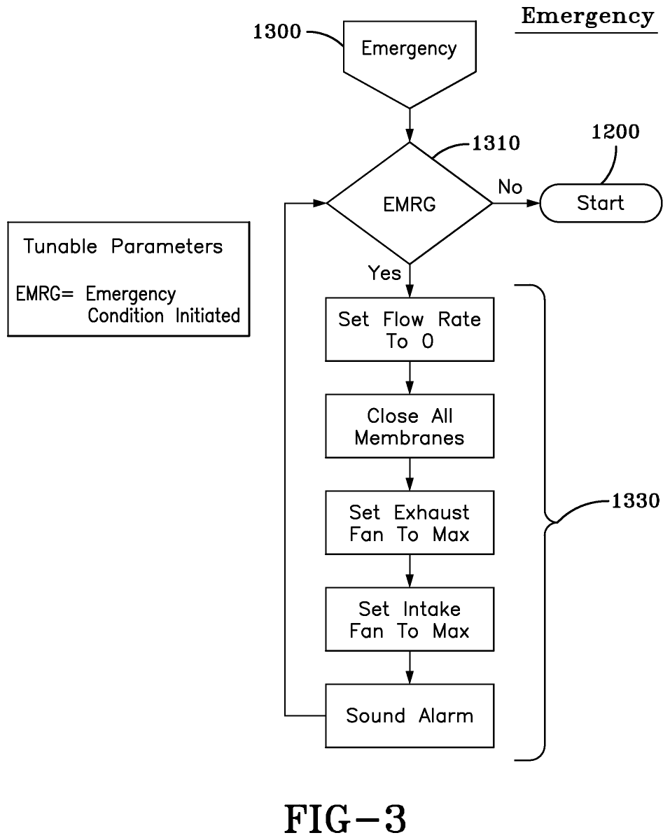

FIG. 3 shows the ACA flowchart for the emergency descent state 1300. The algorithm rechecks the state of the emergency stop button 1310. If it has been released then the algorithm returns to the start condition 1200. Otherwise a series of actions 1330 are taken: the nitrogen generation system flow is stopped by setting the flow controller 700 to zero flow and deactivating all membrane solenoid valves 440; the training enclosure intake fan 200 and exhaust fans 300 are set to their maximum speeds to vent the enclosure as quickly as possible; and visual and audible alarms are activated to alert personnel to the emergency condition. The algorithm then recycles to remain in this loop until the emergency stop button is released 1310.

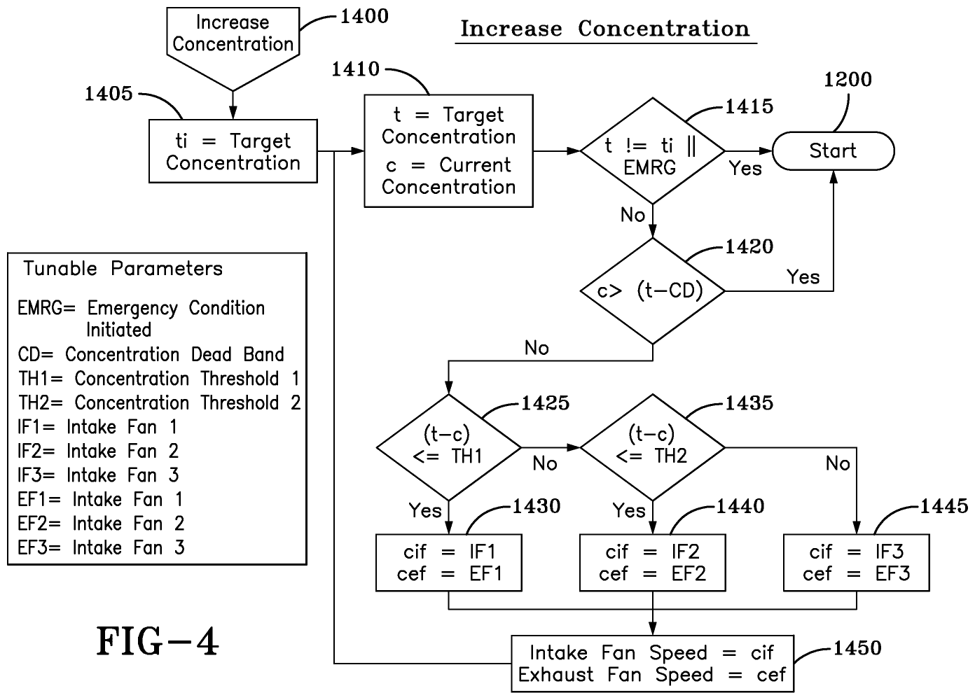

FIG. 4 shows the ACA flowchart for the increase concentration state 1400. Upon entering the state, the initially set target oxygen concentration is stored 1405. The current target and training environment oxygen concentrations are then sampled 1410. If the initial and current target concentrations do not match 1415, indicating the operator has changed the target altitude, or the emergency stop button has been depressed, the algorithm returns to the start condition 1200. If the training environment oxygen concentration is greater than the target concentration minus the dead band value 1420, the target concentration is considered to have been reached and the algorithm returns to the start condition 1200. If the difference between the target and current training environment oxygen concentrations is greater than or equal to a preset high concentration difference threshold 1425, then the intake and exhaust fan speeds are set to high values 1430. If the difference in oxygen concentrations is less than the high concentration difference threshold but greater than or equal to a lower threshold value 1435, then the intake and exhaust fan speeds are set to medium values 1440. If the difference in oxygen concentrations is less than the lower difference threshold value, then the intake and exhaust fan speeds are set to low values 1445. In a preferred embodiment, three fan speed settings are used, creating three simulated altitude descent profiles. In the general case, a larger number of fan speed settings could be used and more complex logic enacted to allow finer control of the simulated descent. The intake and exhaust fan speed controllers are given the selected fan speed values 1450 and the increase concentration loop restarts. Decreasing fan speeds as the target concentration is approached prevents the training environment oxygen concentration from overshooting the target value.

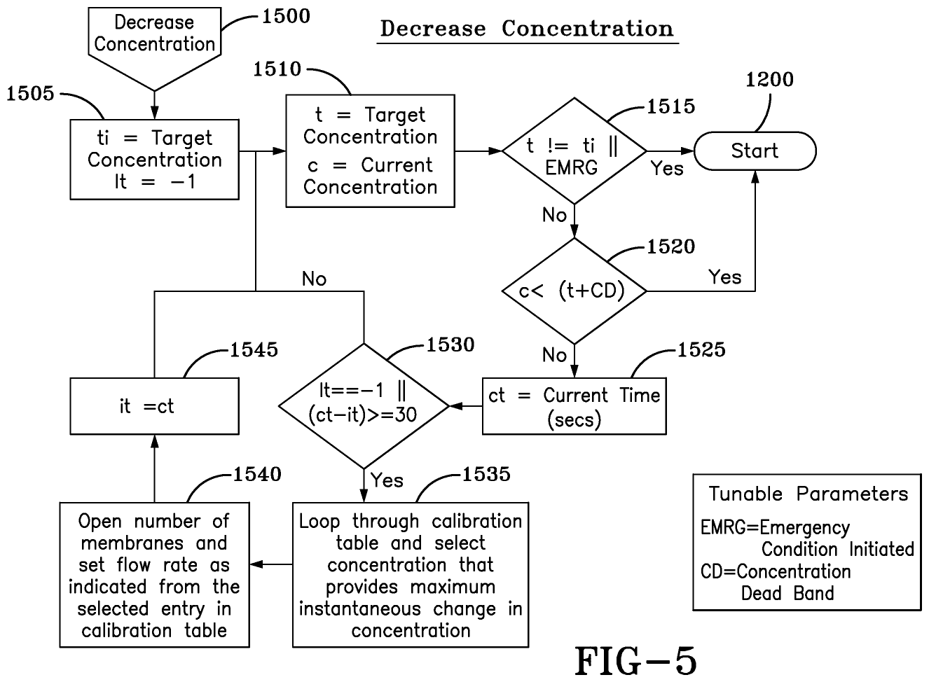

FIG. 5 shows the ACA flowchart for the decrease concentration state 1500. Upon entering the state, the initially set target oxygen concentration is stored and a timer started 1505. The current target and training environment oxygen concentrations are then sampled 1510. If the initial and current target concentrations do not match, indicating the operator has changed the target altitude, or the emergency stop button has been depressed 1515, the algorithm returns to the start condition 1200. If the training environment oxygen concentration is less than the target concentration plus the dead band value 1520, the target concentration is considered to have been reached and the algorithm returns to the start condition 1200. Otherwise, the current time is noted 1525. If this is the first time through the decrease concentration loop or thirty seconds have passed since the timer was started 1530, the algorithm checks a pre-populated performance data array, described below, to determine the number of polysulfone membrane cartridges and air flow to use to reach the target oxygen concentration 1535. If thirty seconds have not yet elapsed since the last time the algorithm examined, the performance data array the decrease concentration loop restarts. The thirty second timer used throughout the ACA is based upon the response time of the oxygen sensors installed in the preferred instance of the normobaric hypoxia trainer, but, in the general case, may be set to any value.

The performance data array is populated during system calibration procedures. During calibration, the oxygen concentration of the nitrogen generation system output flow is measured and recorded for every combination of number of active polysulfone membrane cartridges 430 and air flow rate in increments of a few standard cubic feet per minute. Not all combinations will be possible; the maximum achievable flow rate decreases as the number of cartridges used increases due to the limitation of the maximum compressor output flow. Unachievable combinations are null entries in the performance data array.

The ACA algorithm checks the performance data array during runtime and predicts the instantaneous rate of change of the training environment oxygen concentration that would result, under the current training environment conditions, from each possible combination of output oxygen concentration and flow rate in the array. The algorithm selects the array entry that yields the desired rate of change. This is typically the fastest rate of change, but a lower rate may be chosen to create a particular ascent profile. The control system enables the number of membrane cartridges 430 and sets the air flow rate corresponding to the selected array entry 1540. Finally, the timer is reset 1545 and the decrease concentration loop restarted. In this way the ACA algorithm regularly optimizes the instantaneous rate of change of the training environment oxygen concentration.

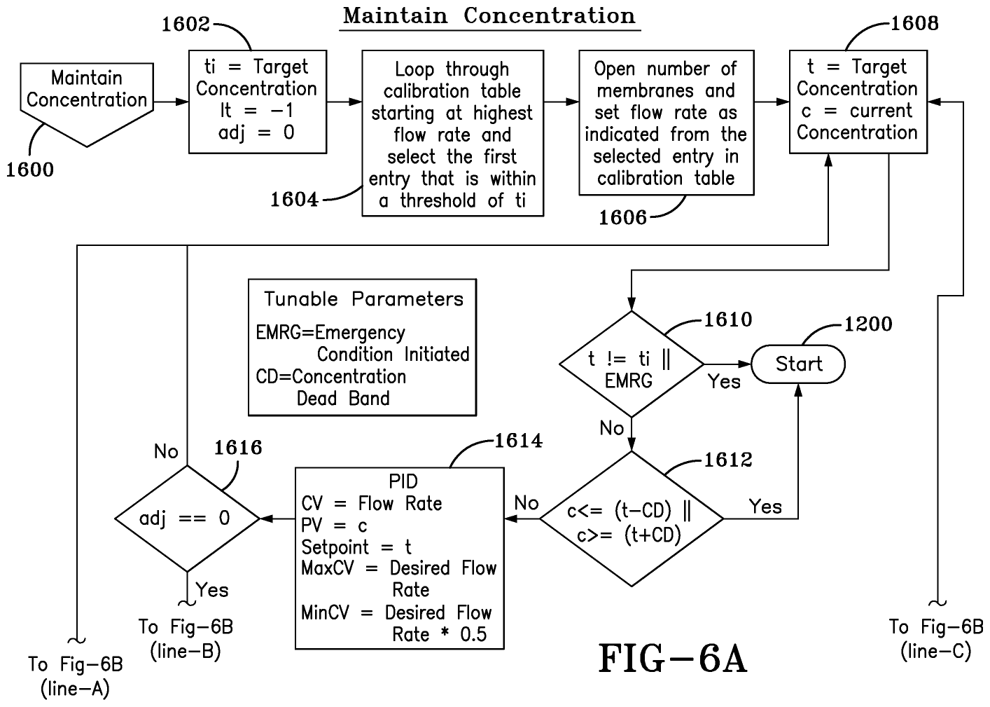

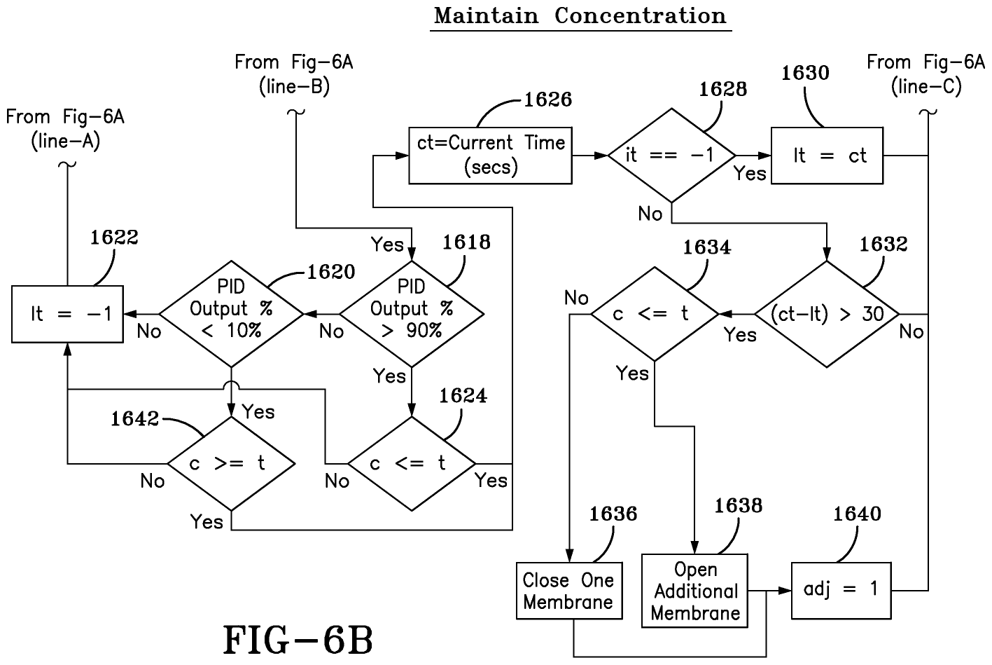

FIGS. 6A and 6B show the ACA flowchart for the maintain concentration state 1600. Upon entering the state, the initially set target oxygen concentration is stored and a timer and adjustment flag substantiated 1602. The ACA then checks the performance data array entries, in order of descending flow rate, selects the first entry encountered that falls within the dead band value of the target oxygen concentration 1604, and activates the indicated number of membranes 1606 by opening those membranes' pneumatic solenoid valves 440. Selecting the highest flow rate possible mitigates the buildup of carbon dioxide by purging the training environment as quickly as possible. The current target and training environment oxygen concentrations are then sampled 1608. If the initial and current target concentrations do not match, indicating the operator has changed the target altitude, or the emergency stop button has been depressed 1610, the algorithm returns to the start condition 1200. If the current training environment oxygen concentration is less than or equal to the target concentration minus the dead band value or greater than or equal to the target concentration plus the dead band value 1612, the training environment is considered to have moved away from the target concentration and the algorithm returns to the start condition 1200 to correct the variance. If the current oxygen concentration is within the target dead band, the algorithm uses a proportional-integral-derivative (PID) control loop 1614 to attempt to keep it there.

A PID controller has three constants that must be tuned: the proportional, integral, and derivative gains. Acceptable values for the PID gains are dependent on the properties of the control system, nitrogen generator and training environment, and, in the preferred embodiment, have been determined through experimentation. The PID controller is allowed to vary the nitrogen generator flow rate in an attempt to drive the current training environment oxygen concentration toward the target concentration 1614. The maximum flow rate available to the PID controller is the flow rate selected by the ACA algorithm from the pre-populated performance data array upon entering the maintain concentration state 1604, while the minimum available flow rate is defined as half that value.

After the PID controller has selected a flow rate, the adjustment flag state is checked 1616. If the flag is raised, the ACA restarts the maintain concentration loop 1600. If the adjustment flag is low, the algorithm examines the flow rate selected by the PID controller. If the flow rate is less than ninety percent 1618 and greater than ten percent 1620 of the flow rate range available to the PID controller, it is judged that the PID controller has enough control range available to be able to maintain the target oxygen concentration. The timer is turned off 1622 and the maintain control loop is restarted at block 1608. If the flow selected by the PID controller is greater than ninety percent of the maximum flow rate range available to the controller 1618 and the current training environment oxygen concentration is still less than or equal to the target concentration 1624, it indicates the current combination of max flow rate and number of membrane cartridges in use can likely not maintain the target concentration under the current training environment conditions. In that case, the current time is noted 1626. If the timer is currently turned off 1628, it is started 1630 and the maintain concentration loop restarts at block 1608. If the timer has been running for less than thirty seconds 1632 the loop is restarted at block 1608. If, however, the timer indicates the ACA has been in this condition for over thirty seconds an adjustment is called for. The current training environment oxygen concentration is compared to the target oxygen concentration 1634. If the current oxygen concentration is higher than the target concentration (the simulated altitude is higher than the target altitude), a polysulfone membrane cartridge solenoid valve 440 is turned off 1636, removing a polysulfone membrane cartridge 430 from the array in use. Removing a cartridge 430 from the array at a constant array flow rate has the effect of speeding the flow of air through each remaining cartridge, decreasing their effectiveness and raising the oxygen concentration in the output flow. The adjustment flag is then raised 1640, indicating the original membrane count has been changed, and the maintain concentration loop restarted at block 1608. In this way the PID controller is given a higher range of oxygen concentrations to work with to attempt to raise the training environment oxygen concentration to the target concentration (decrease altitude).

Similarly, if the flow rate selected by the PID controller in block 1614 is in the bottom ten percent of the flow range available to the controller 1620 and the current training environment oxygen concentration is still greater than the target concentration 1642 (the simulated altitude is lower than the target altitude), it indicates the current combination of max flow rate and number of membrane cartridges may not be able to maintain the target oxygen concentration. If the ACA has been in this state for over thirty seconds 1632, then an additional polysulfone membrane cartridge 430 is added to the array by activating its solenoid valve 440. Adding a polysulfone cartridge to the array while maintaining a constant array flow rate has the effect of slowing the speed of air through each cartridge, increasing their effectiveness and lowering the output oxygen concentration. The adjustment flag is raised 1640 and the maintain concentration loop is restarted at block 1608. In this way, the PID controller is given a lower range of oxygen concentrations to work with to attempt to lower the training environment oxygen concentration to the target concentration (increase altitude).

The preferred embodiment of the ACA maintain concentration state does not allow for a second membrane cartridge array adjustment to be made. If the PID controller still cannot hold the target concentration after a single adjustment the training environment oxygen concentration will eventually move out of the target concentration dead band and the ACA will leave the maintain concentration state to return to the start condition and begin a correction.

In one of the embodiments, the cartridge array may be made to be larger to produce higher air flows. The system and method described here is realized with seven polysulfone membrane cartridges 430 supplied by a 50HP compressor (not shown) but could be expanded to control any reasonable number of cartridges 430 and more powerful compressors to achieve higher air flows and/or finer control of oxygen concentration. Such alternate systems could be used to condition the air of a larger volume, reduce the time required to achieve a simulated altitude or achieve and/or hold a simulated altitude more precisely than the realized invention.

The current invention controls the nitrogen purity of the air output from the polysulfone membrane cartridge array by varying the number of cartridges 430 in use and the air flow through them. The pressure and temperature of the air passed through the polysulfone cartridges 430 are held constant. Finer control of the nitrogen purity could be achieved by placing either or both the air pressure and temperature under Altitude Control Algorithm control as well. Finer control of the nitrogen content would allow a desired simulated altitude or flight profile to be held more accurately.

Personnel within the normobaric hypoxia trainer 10 use a recovery air system that provides them a supply of normal breathable air. Trainees breathe recovery air through flight masks during non-training idle periods or after self-diagnosing hypoxia, while instructors within the chamber breathe from the recovery air system throughout training. Humans utilize only a small portion of the oxygen in their lungs with each breath. Most of the oxygen provided to the trainees through the recovery air system is released into the training enclosure as they exhale. This exhaled oxygen works to lower the effective altitude of the training enclosure. The ACA currently reacts to this effect only after it has measurably altered the oxygen concentration of the training enclosure. However, for training purposes the operators of the NHT are provided with a method of noting when trainees don and remove their recovery masks. The ACA therefore could be made to account for how many persons are exhaling oxygen into the training enclosure at any moment and, using average values of respiration rate, efficiency and lung volume known to the medical community, counteract the oxygen injection before the effect becomes apparent in the simulated altitude. Furthermore, the detection of recovery air use by the trainees could be made automatic rather than relying upon operator input. Another improvement could be made by measuring the actual air flow through the recovery air system, rather than relying upon estimated or average values, to even more accurately gauge and counteract the effect of the recovery air system's oxygen injection before the simulated altitude is perturbed.

When introducing elements of the present invention or the preferred embodiment(s) thereof, the articles “a,” “an,” “the,” and “said” are intended to mean there are one or more of the elements. The terms “comprising,” “including,” and “having” are intended to be inclusive and mean that there may be additional elements other than the listed elements.

Although the present invention has been described in considerable detail with reference to certain preferred embodiments thereof, other embodiments are possible. Therefore, the spirit and scope of the appended claims should not be limited to the description of the preferred embodiment(s) contained herein.

What is claimed is:

1. A normobaric hypoxia trainer comprising:
a training chamber;
an intake fan for allowing ground level air to be introduced into the training chamber;
an exhaust fan for removing air from the training chamber;
a plurality of circulation fans for mixing interior air of the training chamber to create a uniform oxygen concentration within the training chamber;
a nitrogen generation system, the nitrogen generating system including a plurality of membrane cartridges for separating out nitrogen from air;
a compressor for supplying compressed air to the nitrogen generation system;
a pressure regulator for regulating the pressure of the compressed air;
a heater for controlling temperature of the compressed air, the compressed air passing through the polysulphone membrane cartridges such that nitrogen can be separated out from the air; and,
a flow controller for controlling the flow rate of the separated nitrogen into the training chamber.
